(19) United States
(12) Patent Application Publication
(10) Pub. No.: US 2021/0332408 A1
(43) Pub. Date: Oct. 28, 2021

(54) SYSTEMS AND METHODS FOR OPTIMIZATION OF ENZYMES

(71) Applicant: Vader Nanotechnologies Inc., Brooklyn, NY (US)

(72) Inventors: Trévon Gordon, Troy, NY (US); Gordon Fleetwood, Flushing, NY (US); Victor Chu, Alameda, CA (US)

(73) Assignee: Vader Nanotechnologies Inc., Brooklyn, NY (US)

(21) Appl. No.: 17/238,641

(22) Filed: Apr. 23, 2021

Related U.S. Application Data
(60) Provisional application No. 63/015,289, filed on Apr. 24, 2020.

(51) Int. Cl. C12Q 1/18; C12Q 1/04

(52) U.S. Cl. CPC C12Q 1/18; C12Q 1/04

(57) ABSTRACT

Poly and perfluoroalkyl substances (PFAs) are extremely resistant to natural degradation. Described herein are compositions, assays, and methods for generating microorganisms capable of accelerating degradation of these products using a directed evolution strategy. A minimal media including the target carbon source of interest, creates selective pressure for microorganisms capable of degradation. Extremely slow growth rates on these alternative carbon sources is nevertheless measureable using automated image capture and processing pipeline allows one to observe very small changes in cell growth over the course of a very long period of time, thereby identifying microorganisms that could not otherwise be identified.

### CROSS-REFERENCE TO RELATED APPLICATIONS

This application claims benefit under 35 U.S.C. § 119(e) of U.S. Provisional Application No. 63/015,289 filed Apr. 24, 2020, the contents of which are incorporated herein by reference in its entirety.

### FIELD OF THE INVENTION

Provided herein are methods, assays, systems, and compositions related to generation of organisms for degradation of plastics.

BACKGROUND

Plastics and “Forever Chemicals” such as poly and perfluoroalkyl substances (PFAs) are extremely resistant to natural degradation. As a result, these chemicals accumulate in our environment, the food we eat, and our bodies. PFAs, a class of more than 4,000 different chemicals, can be found in everything ranging from household items, fast food wrappers, human blood samples and tap water.

Thermal depolymerization is widely used in plastic recycling, generating light crude oil that can serve as a fuel source or used to generate new plastic material. The cost of the process, currently about $10 per barrel, could be substantially reduced by the pre-digestion of input materials. A further goal is breaking down PFA chemicals into non-toxic products. This will reduce the energy needed for various water purification methodologies, including activated carbon absorption, ion exchange, and reverse osmosis. There is a great need in the art for compositions and methods for accelerating degradation of these products to ward off their destructive accumulation in the environment and organisms.

### SUMMARY OF THE INVENTION

The methods described herein can be used to generate microorganisms for the degradation of plastics. Directed evolution is applied to create new organisms and enzymes that break down chemicals to allow for recycling of plastic and degrade PFAs “forever chemicals”. A key advantage is reducing the energy required for the thermal depolymerization of plastics.

In one aspect, provided herein is an assay comprising:
(a) culturing a population of microorganisms in a minimal media comprising nitrogen and one or more carbon molecules;
(b) capturing a series of images of the microorganisms; and
(c) processing the series of images, wherein processing the series of images identifies one or more phenotypes of interest in the population of microorganisms.

In another aspect, provided herein is a system comprising:
(a) a reservoir for containing a population of microorganisms in a minimal media, wherein the minimal media comprises nitrogen and one or more carbon molecules;
(b) a camera capable of capturing a series of images of the microorganisms; and
(c) a data processing apparatus.

In yet another aspect, provided herein is a culture of a population of microorganisms possessing the one or more phenotypes of interest identified the assay provided herein.

In another aspect, provided herein is a biological agent isolated from the population of microorganisms possessing the one or more phenotypes of interest identified by the assay provided herein.

In another aspect, provided herein is a method for selecting a microorganism for the degradation of waste, the method comprising:
(a) culturing a pre-selected population of microorganisms in a cell culture well, wherein the cell culture well comprises a minimal media comprising a PFA chemical or a plastic in individual culture wells; then
(b) capturing a series of images of the cell culture well;
(c) processing the series of images from step (b), wherein processing the series of images determines an optical density; and
(d) when the optical density of the cell culture increases over a period of time, selecting the population of microorganisms for degradation of waste.

In one embodiment of any of the aspects, the population of microorganisms is subjected to mutagenesis prior to culturing step (a).

In another embodiment of any of the aspects, the mutagenesis comprises random mutagenesis.

In another embodiment of any of the aspects, the mutagenesis comprises homologous recombination.

In another embodiment of any of the aspects, the one or more carbon molecules comprises plastic.

In another embodiment of any of the aspects, the one or more carbon molecule comprises PFAS.

In another embodiment of any of the aspects, capturing a series of images of the microorganisms in step (b) is over a period of at least 1 week, 2 weeks, 3 weeks, 4 weeks, 5 weeks, or more.

In another embodiment of any of the aspects, processing the series of images comprises applying a calibration curve generated by a second-degree polynomial curve from two or more of: a raw image measurement, optical density and cell density values.

In another embodiment of any of the aspects, the one or more phenotypes of interest in the population of microorganisms comprises a growth curve.

In another embodiment of any of the aspects, the one or more phenotypes of interest comprise plastic degradation or PFAS degradation.

In another embodiment of any of the aspects, the biological agent is a peptide or protein.

In another embodiment of any of the aspects, the biological agent is a nucleic acid.

In another aspect, provided herein is a media comprising nitrogen and one or more carbon molecules. In various embodiments, the media includes one or more plastics. Non-limiting examples include, Polyethylene (PE), Polyethylene Terephthalate (PET), Polyvinyl Chloride (PVC), Polypropylene (PP), Polystyrene (PS), Polylactide Acid (PLA), Polycarbonate (PC), Acrylic (PMMA), Acetal (Polyoxymethylene, POM), Nylon (PA), ABS (Acrylonitrile Butadiene Styrene).

In another embodiment of any of the aspects, the one or more carbon sources include a PFA chemical. In another embodiment of any of the aspects, the PFA chemical is selected from the group consisting of: Perfluorooctanoic acid (PFOA), Perfluoroctanesulfonic acid (PFOS), Perfluorohexane sulfonate (PFHxS), and Perfluorononanoic acid (PFNA)..

In another embodiment of any of the aspects, the microorganisms are pre-selected for culturing.

In another embodiment of any of the aspects, the pre-selected population of microorganisms are the same species or strain of microorganisms.

In another embodiment of any of the aspects, the pre-selected population of microorganisms are different species or strains of microorganisms.

### BRIEF DESCRIPTION OF THE FIGURES

This application file contains at least one drawing executed in color. Copies of this patent application publication with color drawings will be provided by the Office upon request and payment of the necessary fee.

FIG. 1A Process overview. FIG. 1B Testing of the remote monitoring system, showing time-lapse and associated time series data attached. FIG. 1C is a detailed view of a tested plate.

FIG. 2A E. coli cells settle form biofilms and slight growth is observed. FIG. 2B Day 4 cells grow better on ethylene glycol. Selected as the starter organisms for the next round of DE. FIG. 2C shows multi-well plates are used to scale from 11 experiments to roughly 200 experiments for a fully automated process of identifying microorganisms that degrade plastics and/or PFA chemicals.

FIG. 3 shows a custom designed a camera that can be 3D printed and attached on the Opentrons robot to improve data collection.

FIG. 4 shows the camera attached on the Opentrons robot that takes images of the deck.

FIG. 5 shows edged detection that can be used for well detection. Once a well is detected, individual wells (experiments) are cropped using the circle crop and run through the image processing pipeline.

FIG. 6 shows sample output processing for Maximum PFNA. Left: Images of the experiment at times T=0 hrs, T=10 hrs, and T=80 hrs before image processing and data visualization Right: the result of image processing and data visualization reveals that increasing PFNA concentration slows bacterial growth.

FIG. 7 shows sample output processing for Minimum Nutrients. Left: Images of the experiment at times T=0 hrs, T=20 hrs, and T =80 hrs before image processing and data visualization Right: the result of image processing and data visualization reveals that reducing nutrient concentration reduces maximum cell concentration.

FIG. 8 shows an overview of the strain development process for a PFAS degrading strain or population of strains. This cyclical process is subjected to directed evolution for desired PFAS degradation properties.

FIG. 9 shows an overview of the strain development process for a plastic degrading strain or population of plastic-degrading strains. Plastic-degrading strains can also be subjected to directed evolution for desired degradation properties of plastics.

FIGS. 10 and 11 illustrate experimental results showing significant changes in PFNA concentration after incubation with Pseudomonas strains.

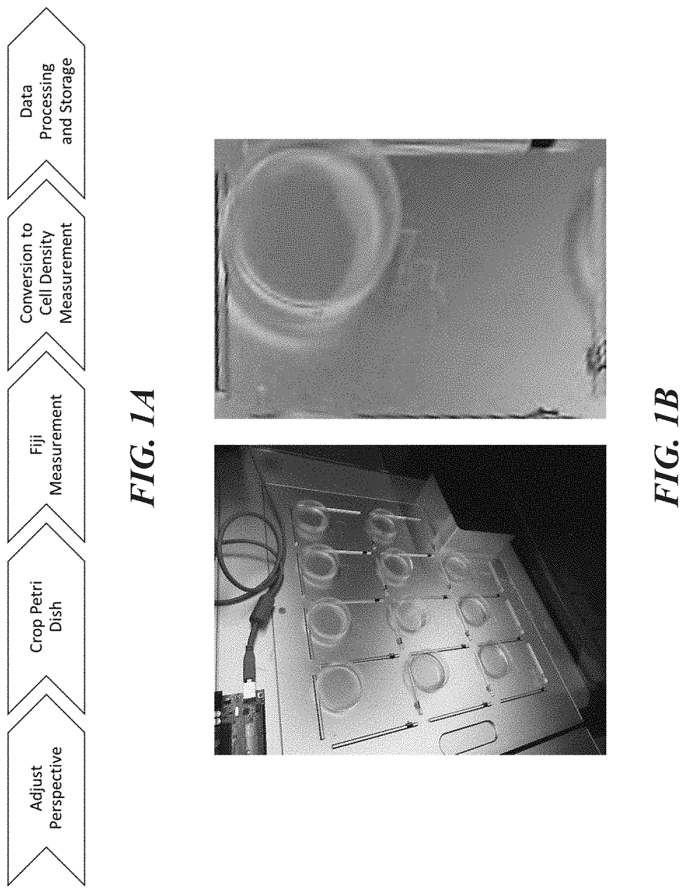

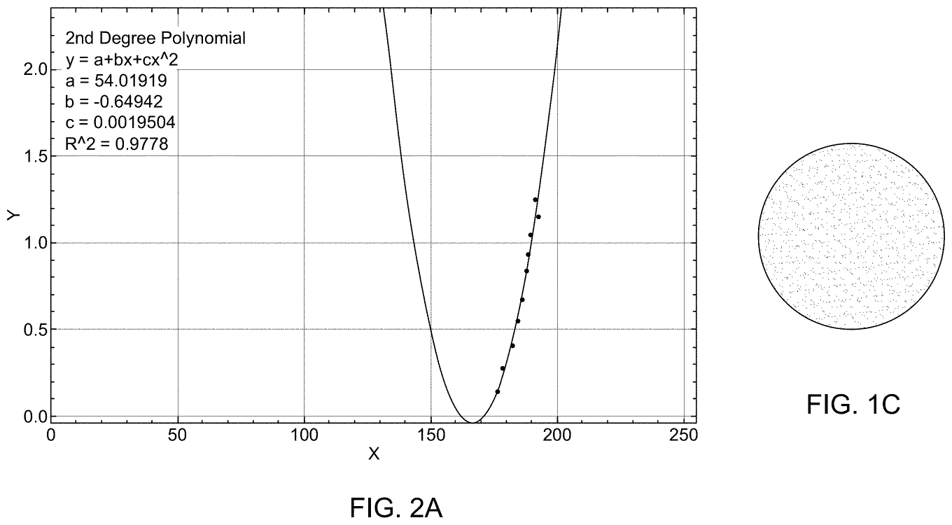

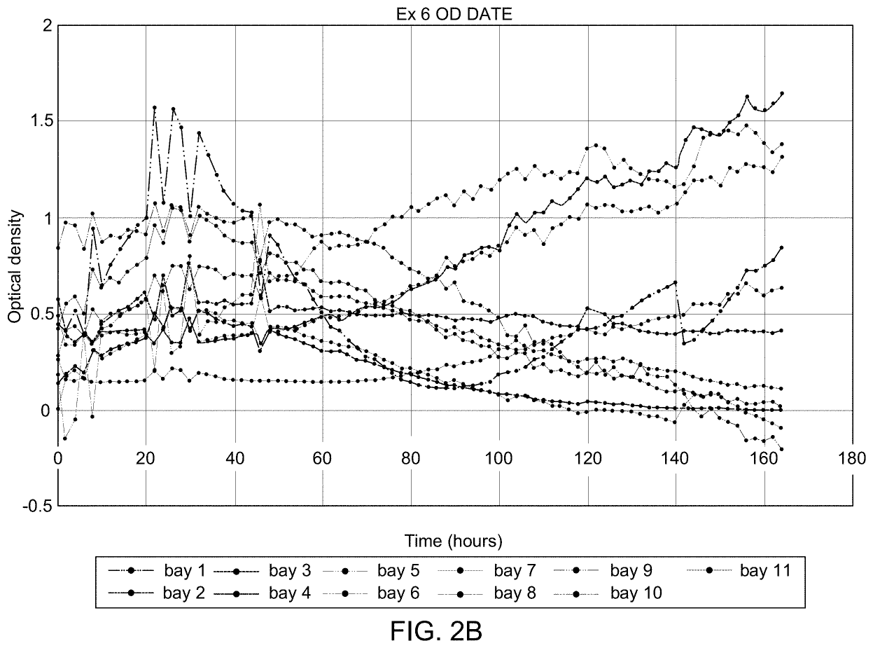

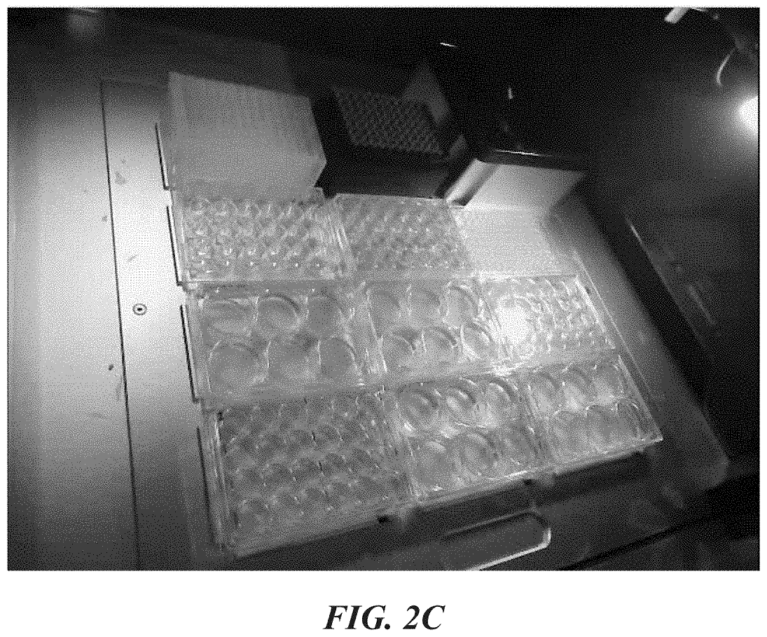

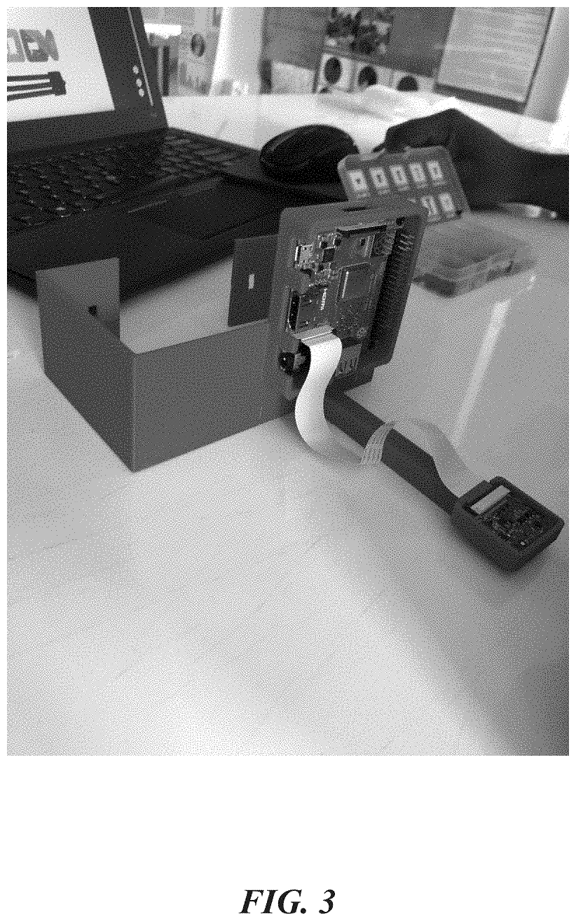

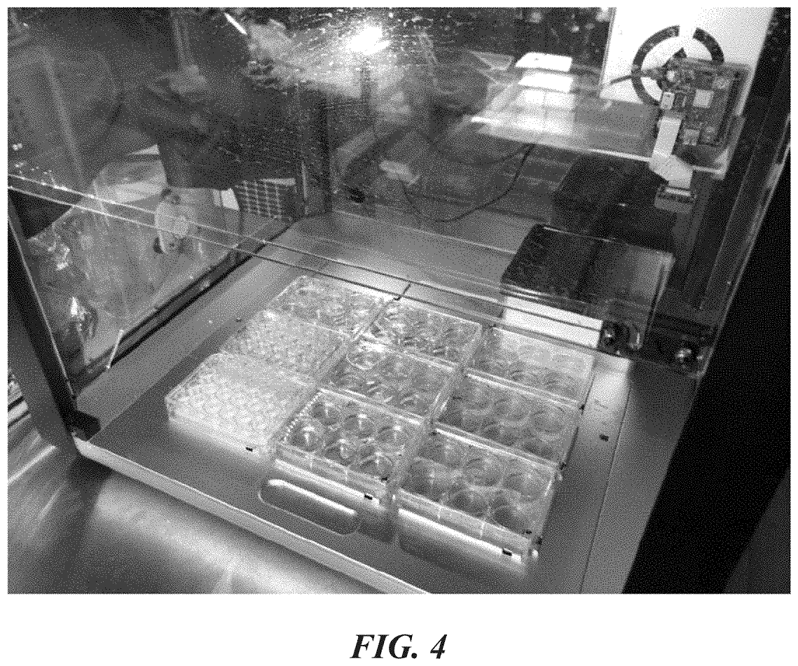

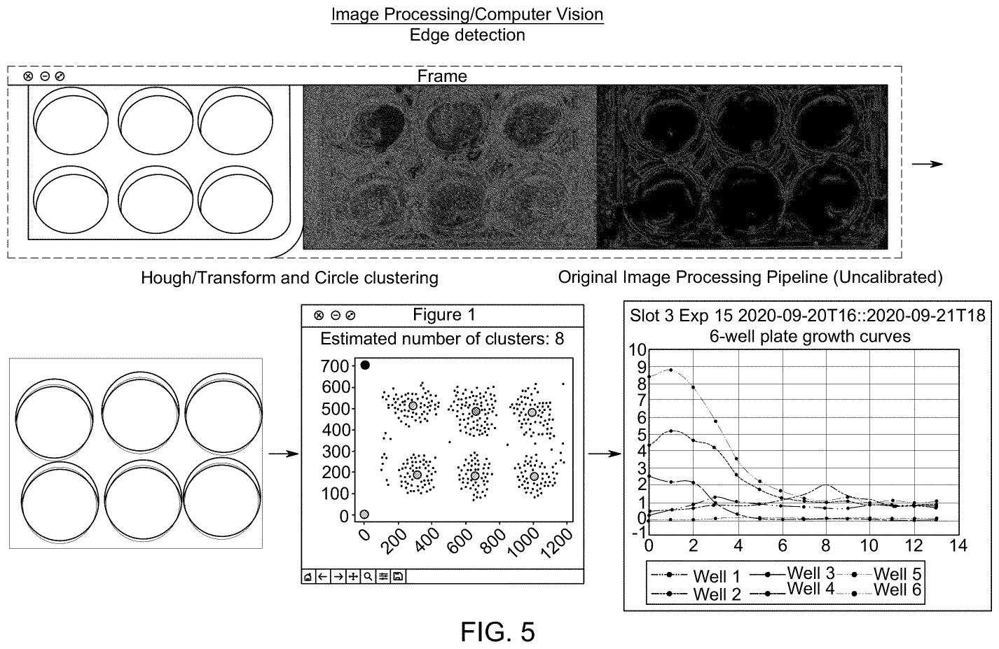

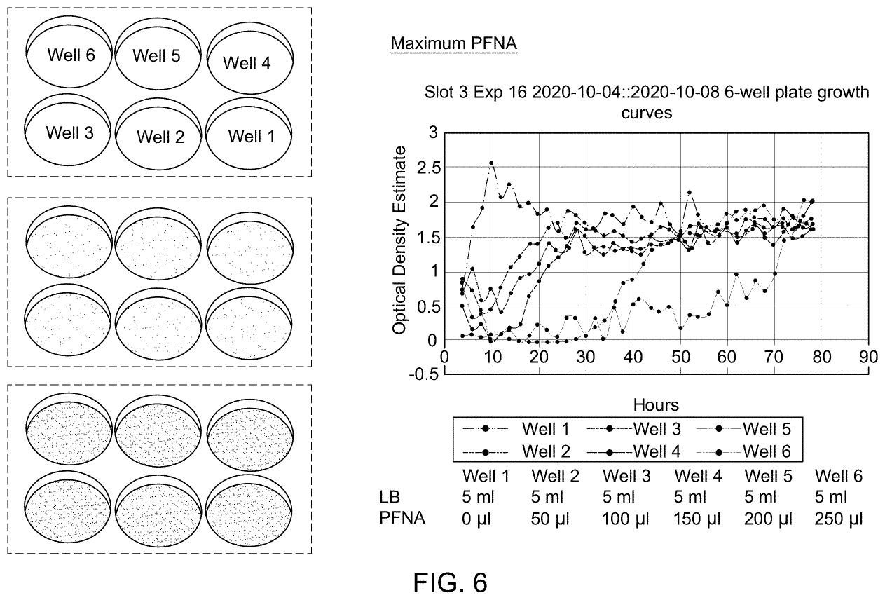

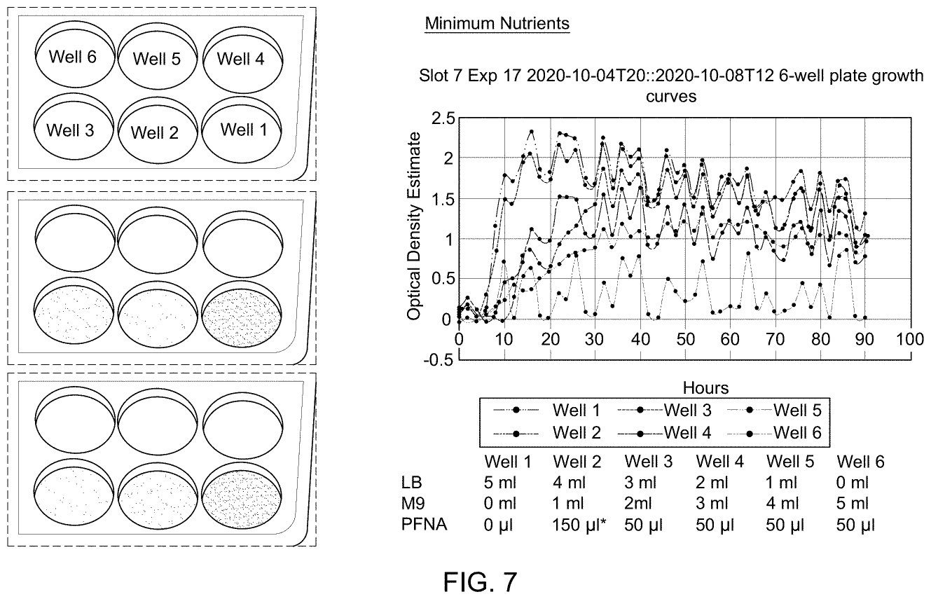

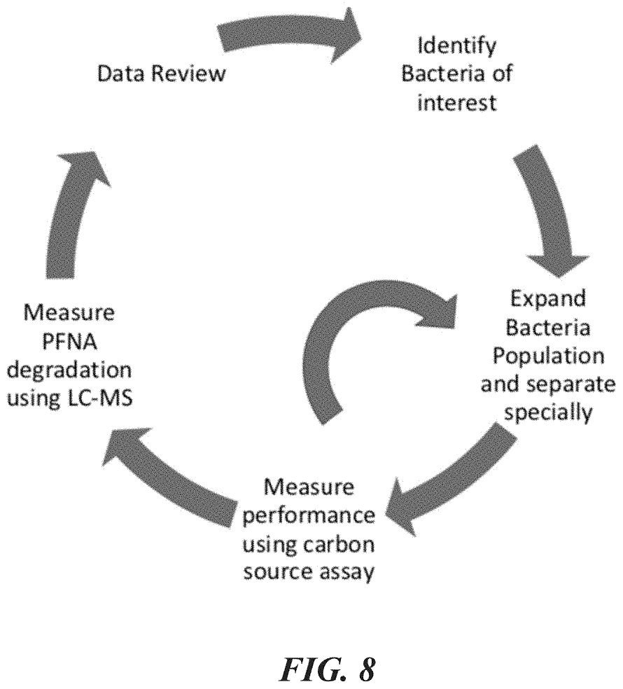

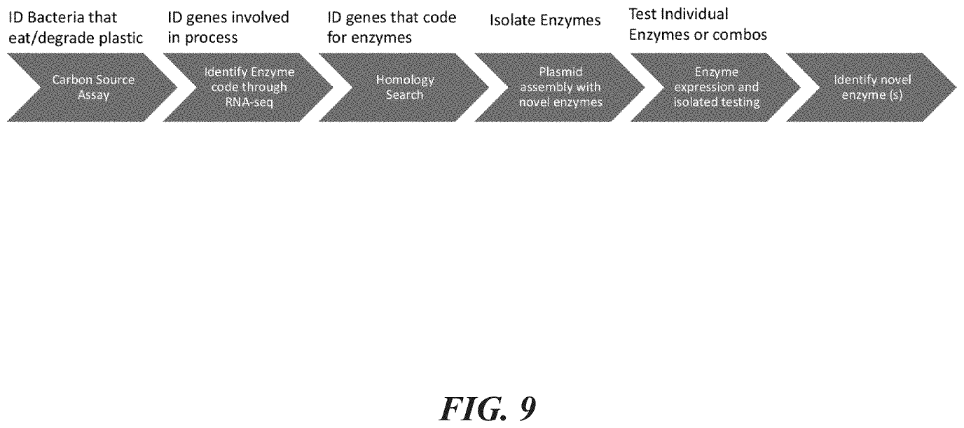

### DETAILED DESCRIPTION OF THE INVENTION

The compositions, methods, and assays provided herein are based, in part, on the discovery that a minimal media platform for selective pressure of carbon source subsistence can be used to identify microorganisms that can degrade PFAS. Thus, the assays, compositions, and methods provided herein can be used to identify microorganisms than can reduce plastic and PFA chemical waste from the environment.

One of the major problems facing our world today is the substantial quantities of plastics piling up in landfill sites and in natural habitats worldwide, generating increasing environmental problems. Even degradable and biodegradable plastics may persist for decades. Furthermore, plastic products and perfluoroalkyl (PFA) have been found to contaminate soils, ground water, water sources (lakes, rivers, oceans, etc.) and the air causing major health and safety risks for animals, plants, and ecosystems.

While conventional techniques at managing waste have been applied, they have not successful. As an alternative, microorganisms can provide an avenue for efficiently breaking down plastic wastes or scavenging PFA chemicals and plastic from contaminated environments.

In particular, conventional directed evolution of microorganisms involves discrete cycles of mutagenesis, transformation or in vitro expression, among others and selecting for the property of interest, including for example, microorganisms capable of degrading plastics or PFAS. In principle, microorganisms forced to subsist solely on plastic or PFAS carbon sources would experience selective pressure allowing for identification of microorganisms of interest, along with evolved biological macromolecules associated with application of such selective pressure.

Laboratory evolution has generated microorganisms and biomolecules with desired properties. However, conventional laboratory evolution strategies rely on discrete mutation-selection rounds, with a directed evolution typically requires days or longer with frequent intervention by a researcher for a single round. This makes complex evolution processes impractical to perform in the laboratory. In all instances of applying directed evolution, successful evolution is strongly dependent on the total number of rounds performed. The labor and time intensive nature of discrete directed evolution cycles limit many laboratory evolution efforts to a modest number of rounds. Automation has been applied to streamline this process, including for example, automated cycles of transformation and recombination to generate targeted diversity in E. coli through automated cycles of transformation and recombination.

However, when considering application of the aforementioned conventional techniques to plastic or PFAS degradation, labor and time cost becomes prohibitive even with the aid of automation. The provided plastic or PFAS sources are composed of low energy carbon-carbon bonds. When attempting to develop organisms to consume these plastics, this low energy source ultimately translates to extremely slow growth rates when such microorganisms are to subsist on these alternative carbon sources. As a result, doubling time goes from minutes to days. Therefore, it takes days if not weeks to see any discernable changes in the microorganism population through this screening method. This makes any conventional attempt as directed evolution wholly impractical. Any attempt to solve the aforementioned problem must provide a precise way to monitor very small changes in cell growth that will occur over the course of a very long period of time.

Briefly, the Inventors' technology provided herein involves use of a minimal media platform for selective pressure of carbon source subsistence and custom imaging pipeline. More specifically, an assay involves growing microorganisms on minimal media supplemented with the carbon source of interest, further including mutagenic agents to spur genetic diversity. The minimal media only contains trace salts and a nitrogen source, but no carbon source. As microorganisms require a carbon source to grow, one supplements the media with the carbon source of interest for microorganism ingestion and metabolic breakdown. This carbon source can be plastic, PFAS chemicals, or other difficult to break down chemicals. The combination of these carbon source, minimal media and mutagenesis, exerts selective pressure is used to identify organisms with the desired metabolic properties.

As the phenotypic properties of surviving microorganisms is difficult to detect (e.g., extremely slow growth profile) emerging over very long time periods (e.g., weeks), automation is utilized to capture images of microorganisms every 2 hours for approximately 2 weeks. Image capture is then processed by proprietary image analysis software to yield a growth curve.

The growth curve can be used to select for the microorganisms and consortia or microorganisms with the desired PFA or plastic degradation properties.

### Sources of Microorganisms and Culture Conditions:

The assays, methods and compositions provided herein comprise or utilize a population of microorganisms cultured in a minimal media that can be screened for PFA chemical and plastic degradation properties. The starting preparation of microorganisms are pre-selected for the assay and methods provided herein without contamination by other microorganisms. In some embodiments, the pre-selected population of microorganisms are the same species or strain of microorganisms. In some embodiments, the pre-selected population of microorganisms are different species or strains of microorganisms.

The term “microorganisms” or “microbes” as used herein, refers to microorganisms that confer a benefit. In some embodiments, the microorganisms are viable. In some embodiments, the microorganisms are non-pathogenic microorganisms (e.g., not associated with a known human disease). Non-limiting examples of microorganisms that can be used in the assays, compositions, and methods provided herein include bacteria, fungi, algae, worms, and parasites. Non-limiting examples of bacteria that can be used in the assays, methods, and compositions provided herein include Acidimicribioum sp. Strain A6, Acidovorax sp., Alcaligens Faecalis. Amycolatopsis sp., Bacillus Subtilis, Bacillus, Brevibacillus borstelensis, Caenibacterium thermophilum, Clostridium acetobutylicum, Clostridium botulinum, Corynebacterium Comamonas acidovorans, Clostridium botulinum, Dehalococcoides Diplococcus sp., Enterobacter, Enterobacter agglomerans, Escherichia, E. coli, Enterococci Ideonella sakaiensis, Lactobacillus, Micrococcus sp., Moraxella sp., Nocardia farcinica, Nitrobacter spp., Nitrosomonas spp., Ochrobactrum, Pichia Pastoris, Pseudomonas, Pseudomonas chlororaphis, Pseudomonas fluorescens, Pseudomonas aeruginosa Pseudomonas putida, Pseudomonas indica, Pseudomonas lemoignei, Pseudomonas stutzeri, Rhodococcus, Ralstonia pikettii, Salmonella, Staphylococcus, Streptococcus, Streptomyces, Shigella, Schlegelella thermodepolymerans, Thiobacillus spp., Listeria, Firmicutes, and Proteobacteria.

Additional examples of microorganisms that can be beneficial for waste degradation are further discussed, e.g., in Puglisi, E., Romaniello, F., Galletti, S. et al. Selective bacterial colonization processes on polyethylene waste samples in an abandoned landfill site. Sci Rep 9, 14138 (2019); Moharir, R. V. & Kumar, S. “Challenges associated with plastic waste disposal and allied microbial routes for its effective degradation: A comprehensive review.” J Clean Prod 208, 65-76 (2019); Pathak, V. M. & Navneet. “Review on the current status of polymer degradation: a microbial approach.” Bioresour Bioprocess 4 (2017); Zettler, E. R., Mincer, T. J. & Amaral-Zettler, L. A. “Life in the “plastisphere”: microbial communities on plastic marine debris.” Environ Sci Technol 47, 7137-7146 (2013); and Bryant, J. A. et al. “Diversity and Activity of Communities Inhabiting Plastic Debris in the North Pacific Gyre.” mSystems 1 (2016). the contents of each of which is incorporated herein by reference in their entireties.

Microbial mechanisms of carbon source metabolism are known in the art and further described, e.g., in Lewis et al. “Engineering artificial communities for enhance FTOH degradation.” Science of the Total Environment 572, 935-942, (2016), the contents of which is incorporated herein by reference in their entireties.

The microbial species included in the assays, compositions, and methods provided herein can be (1) isolated directly from a specimen or taken from a banked stock, (2) optionally cultured on a nutrient agar or broth that supports growth to generate viable biomass, and (3) the biomass optionally preserved in multiple aliquots in long-term storage.

The microorganisms provided herein can be obtained from various sources, e.g., commercially (e.g., the American Type Culture Collection (ATCC)), a landfill, a soil sample, a subject, or a water source.

The microorganisms provided herein can be isolated according to methods known in the art, e.g., those described in Madigan M, Martinko J, eds. (2005). Brock Biology of Microorganisms (11th ed.). Prentice Hall. ISBN 0-13-144329-1, the content of which is incorporated herein by reference its entirety.

### Mutagenesis of Microorganisms

In one embodiment of any of the aspects provided herein, the population of microorganisms is subjected to mutagenesis. In some embodiments, the pre-selected population of microorganisms are engineered to comprise at least one mutation in their genomic DNA.

For example, engineered microbes include microbes harboring i) one or more introduced genetic changes, such change being an insertion, deletion, translocation, or substitution, or any combination thereof, of one or more nucleotides contained on the bacterial chromosome or on an endogenous plasmid, wherein the genetic change can result in the alteration, disruption, removal, or addition of one or more protein coding genes, non-protein-coding genes, gene regulatory regions, or any combination thereof, and wherein such change can be a fusion of two or more separate genomic regions or can be synthetically derived; ii) one or more foreign plasmids containing a mutant copy of an endogenous gene, such mutation being an insertion, deletion, or substitution, or any combination thereof, of one or more nucleotides; and iii) one or more foreign plasmids containing a mutant or non-mutant exogenous gene or a fusion of two or more endogenous, exogenous, or mixed genes. The engineered microbe(s) can be produced using techniques including but not limited to site-directed mutagenesis, transposon mutagenesis, knock-outs, knock-ins, polymerase chain reaction mutagenesis, chemical mutagenesis, ultraviolet light mutagenesis, transformation (chemically or by electroporation), phage transduction, or any combination thereof.

In some embodiments, the mutagenesis comprises random mutagenesis. In some embodiments, the mutagenesis comprises homologous recombination. In some embodiments, the mutagenesis is UV mutagenesis. In some embodiments, the mutagenesis is DNA shuffling. In some embodiments, the mutagenesis is performed using a CRISPR cas system, zinc finger nucleases (ZFNs), transcription activator-like effector nucleases (TALENs), or meganucleases.

Methods of mutagenesis are known in the art and further described, e.g., Sambrook et al., Molecular Cloning: A Laboratory Manual, Cold Spring Harbor Press (1989); Wells et al., Gene 34: 315-23 (1985)); Gilliam et al., Gene 12: 129-137 (1980); Zoller and Smith, Methods Enzymol. 100: 468-500 (1983); Dalbadie-McFarland et al., Proc. Natl. Acad. Sci. (U.S.A). 79: 6409-6413 (1982); Scharf et al., Science 233: 1076-1078 (1986); Higuchi et al., Nucleic Acids Res. 16: 7351-7367 (1988)); Stemmer (1994) PNAS 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) PNAS 94:4504-4509; Crameri et al. (1998) Nature 391:288-291; and U.S. Pat. Nos. 5,605,793, 5,837,458, and 9914939B2.

The presence of a mutation in the microorganism genome can be determined by methods known in the art, e.g., PCR amplification or sequencing techniques.

### Minimal Medium Compositions

In some embodiments of any of the aspects, the microorganisms are cultured in a minimal media. In various embodiments, the media includes one or more of: one or more trace salts, one or more nitrogen sources and one or more carbon molecules.

In some embodiments, the one or more carbon molecules comprises plastic.

In some embodiments, the one or more carbon molecule comprises PFAS.

In various embodiments, the media includes one or more trace salts. In various embodiments, trace salts include Disodium Phosphate (sodium phosphate dibasic), Monopotassium phosphate (potassium phosphate monobasic), Sodium Chloride, Calcium Chloride. In various embodiments, trace salts can also include Copper Sulfate Pentahydrate, Ferric Citrate, Ferric Chloride (Iron(III) Chloride), Ferric Sulfate (Iron (III) Sulfate), Magnesium Sulfate Heptahydrate, Manganese Sulfate Monohydrate, Potassium Chloride, Dipotassium phosphate, Vitamin B1 (thiamine), Sodium Acetate, Sodium Molybdate Dihydrate, Sodium Nitrate, Sodium Tetraborate, Zinc sulfate Heptahydrate, p-Aminobenzoic acid, Niacin, Pyridoxin HCl, Riboflavin, Thiamin HCl, Choline HCL, d-Biotin (or Biotin in general), Adenine HCL L-Methionine, L-Lysine or Arginine, Riboflavin, Yeast Extract, Peptone, Tryptone, Malt Extract.

In various embodiments, the media includes one or more nitrogen sources. In various embodiments, nitrogen sources include Ammonium Chloride, Ammonium Sulphate, Ammonium Nitrate, Yeast Extract, Peptone, Nutrient Broth, Meat Extract and Tryptone.

In various embodiments, the media includes one or more plastics. Non-limiting examples include, Polyethylene (PE), Polyethylene Terephthalate (PET), Polyvinyl Chloride (PVC), Polypropylene (PP), Polystyrene (PS), Polylactide Acid (PLA), Polycarbonate (PC), Acrylic (PMMA), Acetal (Polyoxymethylene, POM), Nylon (PA), ABS (Acrylonitrile Butadiene Styrene). Generally, any crystalline polymer that is durable (e.g., high melting point, etc.) derived from crude oil is compatible with the platform. In various embodiments, the one or more carbon sources is in a film, nurdles, or powder.

In various embodiments, the one or more carbon sources include a perfluoroalkyl (PFA) chemical, which includes but is not limited to: Perfluorooctanoic acid (PFAS), Perfluoroctanesulfonic acid (PFOS), Perfluorohexane sulfonate (PFHxS), Perfluorononanoic acid (PFNA). Additional PFA chemicals and their safety risks to humans and the environment are described, e.g., on the world wide web via the Environmental Protection Agency webpage, in an article titled “Basic Information on PFAS” <epa.gov/pfas/basic-information-pfas>.

In various embodiments, the media includes a precursor molecule of plastic or PFAS with or without the PFAS chemical also in solution. Precursors are known in the art. See, e.g., U.S. Pat. No. 10,954,144 B2, the content of which is incorporated herein by reference in its entirety.

Exemplary solutions for microorganism culture are provided in Tables 1-2.

TABLE 1
M9 Stock Solution

| Component | Addition | |
|---|---|---|
| Sodium Phosphate Dibasic | 15 | g |
| Potassium Phosphate Monobasic | 7.5 | g |
| Ammonium Chloride | 2.5 | g |
| Sodium Chloride | 1.25 | g |
| Calcium Chloride | 9 | mg |
| DI water | 1 | Liter |

DI water is added to a final volume of 1 L. Autoclaved for 15 min @ 121 C.

TABLE 2
PFNA Stock Solution

| Component | Addition | |
|---|---|---|
| Sodium Phosphate Dibasic | 0.120 | g |
| DI water | 1 | Liter |

In some embodiments, the final PFNA concentration is at least 0.01 milligram/Liter (mg/L), at least 0.05 mg/L, at least 0.1 mg/L, at least 0.5 mg/L, at least 1 mg/L, at least 2 mg/L, at least 3 mg/L, at least 4 mg/L, at least 5 mg/L, at least 6 mg/L, at least 7 mg/L, at least 8 mg/L, at least 9 mg/L, at least 10 mg/L, at least 11 mg/L, at least 12 mg/L, at least 13 mg/L, at least 14 mg/L, at least 15 mg/L.

### Assays, Systems, and Methods

Provided herein is an assay, system and method for identifying a phenotype of interest in a population of microorganisms.

In one aspect, the assay provided herein comprises:
(a) culturing a population of microorganisms in a minimal media comprising nitrogen and one or more carbon molecules;
(b) capturing a series of images of the microorganisms; and
(c) processing the series of images, wherein processing the series of images identifies one or more phenotypes of interest in the population of microorganisms.

In another aspect, provided herein is a system comprising:
(a) a reservoir for containing a population of microorganisms in a minimal media, wherein the minimal media comprises nitrogen and one or more carbon molecules;

The assay provided herein can operate in automated high-throughput or manual modes. The assay can be conducted using at least one control. For example, a negative control culture that does not contain a population of microorganisms or a population of microorganisms that do not degrade PFA chemicals. A positive control can also be used, e.g., a culture of microorganisms known to degrade plastics or a culture of microorganisms that are not grown in the minimal media comprising PFA chemicals or plastics.

The carbon source and other nutrients can be supplied by the system provided herein in a batch, fed-batch, or continuous manner and the vessels environmental conditions (flow rates, temperatures, pH, dissolved oxygen, agitation speed, etc.) can be monitored and controlled in automated mode.

In some embodiments, the reservoir is a bioreactor, e.g., those described on the world wide web at Principles of Food and Bioprocess Engineering (FS 231) Bioreactors and Fermentation <projects.ncsu.edu/project/foodengineer/231/notes/bioreactor/Bioreactors-and-Fermentation-Notes. pdf>

Phenotypes of interest can be determined by the images of the microorganisms grown in the minimal media provided herein. Non-limiting examples of a phenotype of interest can include increased cell density, increased cell growth, increased turbidity, color changes, or chemical changes (e.g., the presence of PFA chemical metabolites). In some embodiments, the phenotype of interest in the population of microorganisms is a growth curve. In some embodiments, the one or more phenotypes of interest comprise plastic degradation or PFAS degradation.

In some embodiments, capturing a series of images of the microorganisms is over a period of at least 1 week, 2 weeks, 3 weeks, 4 weeks, 5 weeks, or more. Time-lapse image analysis methods are described, e.g., Mizuno et al. Scientific Reports (2019).

Images can be taken on certain automation systems (e.g., Opentron robots). For example, a processing system converts the single image of the deck into flat facing images of each of the bays on the robot. Image measurements can be converted to optical density or cell density measurements by a calibration curve. The curve is generated by measuring known optical density values in a petri dish to yield a corresponding raw image measurement value. Calibration curve is then constructed by plotting the raw image measurement values (x values) against the optical density or cell density values (y values) and then fitting these points with a second-degree polynomial curve.

In some embodiments, processing the series of images comprises applying a calibration curve generated by a second-degree polynomial curve from two or more of: a raw image measurement, optical density and cell density values.

Optical density or turbidity changes as bacteria grow in solution. In the automated carbon source assay, this growth is due to catabolism of the carbon source and other important micronutrients in solution. See, e.g., Carlson et al., “Selective carbon sources influence the end products of microbial nitrate reparation.” ISME Journal (2020); and Kwon et al. “Biodegradation of perfluorooctandsulfonate (PFOS) as an emerging contaminant” Chemosphere 109 221-225, (2014).

An increase in the optical density in a reservoir or cell culture well comprising a population of microorganisms can indicate an increase in cell density and the degradation of the plastic or PFA chemical that is present in the minimal media as compared with the starting time point. When optical density increases, the microorganisms can then be selected for waste degradation applications, undergo further mutagenesis, and/or be used in combination with other useful microorganisms for additive effects on PFA chemical and plastic waste degradation.

Parallel processing can be used to speed up or enhance the processing of the thousands of images created during the experiment. Furthermore, phenotypes of interest can be verified or validated by methods known in the art, e.g., mass spectrometry or PCR techniques known in the art.

### Methods of Selecting Microorganisms for Waste Degradation and Composition Thereof

In another aspect, provided herein is a culture of a population of microorganisms possessing the one or more phenotypes of interest identified the assay provided herein.

In another aspect, provided herein is a method for selecting a microorganism for the degradation of waste, the method comprising:

(a) culturing a pre-selected population of microorganisms in a cell culture well, wherein the cell culture well comprises a minimal media comprising a PFA chemical or a plastic in individual culture wells; then

(b) capturing a series of images of the cell culture well;

(c) processing the series of images from step (b), wherein processing the series of images determines an optical density; and

(d) when the optical density of the cell culture increases over a period of time, selecting the population of microorganisms for degradation of waste.

In another aspect, provided herein is a biological agent isolated from the population of microorganisms possessing the one or more phenotypes of interest identified by the assay provided herein. Methods of isolating biological agents from a microorganism are known in the art, e.g., polypeptide purification. In some embodiments, the biological agent is a peptide or protein. In some embodiments, the biological agent is a nucleic acid.

In some embodiments, the population of microorganisms selected for waste degradation can decrease the level (e.g., concentration) of a PFA chemical or plastic in the culture by at least 10%. In other embodiments, the level of a PFA chemical or plastic is decreased by at least 20%, at least 30%, at least 40%, at least 50%, at least 60%, at least 70%, at least 80%, at least 90%, at least 1-fold, at least 2-fold, at least 5-fold, at least 10-fold, at least 50-fold, at least 100-fold, at least 500-fold, at least 1000-fold, at least 5000-fold, at least 10,000-fold, at least 15,000-fold or at least 20,000-fold over the level of PFA chemical or plastic of a microorganism that does not degrade PFA or plastic or a reference level.

In some embodiments, the method further comprises culturing the selected population(s) of microorganisms in a bioreactor. In some embodiments, the method further comprises formulating the selected population(s) of microorganisms for dispersal in waste material or water sources.

In another aspect, provided herein is a composition for waste degradation, the composition comprising:

(a) a preparation of a microbial consortium of microorganisms that possess one or more phenotypes of interest identified by the assay provided herein; and

(b) a carrier.

In some embodiments, the phenotype is PFA chemical or plastic degradation. In some embodiments, the microorganisms are bacteria.

In some embodiments, the microbial consortium of microorganisms form a biofilm. Biofilms are described, e.g., in Donlan R M. Biofilms: microbial life on surfaces. Emerg Infect Dis. 2002;8(9):881-890, the contents of which is incorporated by reference in its entirety.

In some embodiments, the carrier is selected from a medium, a pharmaceutically acceptable carrier, a gel, a powder, a soil, a compost pellet, a fertilizer, a tablet, a solid support.

In some embodiments, the composition is mixed with an agriculturally compatible carrier. The synthetic preparation can also comprise a carrier, such as diatomaceous earth, clay, or chitin, which act to complex with chemical agents, such as control agents. The carrier can be a solid carrier or liquid carrier, and in various forms including microspheres, powders, emulsions and the like. The carrier may be any one or more of a number of carriers that confer a variety of properties, such as increased stability, wettability, or dispersability.

In some embodiments, the microorganisms identified by the assay provided herein are lyophilized and reconstituted prior to use for waste degradation. In some cases, the reconstitution is by use of a diluent, e.g., ethanol, glycerol, water.

Additional non-limiting examples of suitable carriers include, but are not limited to, lactose, starch, glucose, methyl cellulose, magnesium stearate, mannitol, sorbitol, starch, gelatin, glycerol, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol, sodium benzoate, sodium acetate, sodium chloride, tackifier, a microbial stabilizer, a fungicide, an antibacterial agent, an herbicide, a nematicide, an insecticide, a plant growth regulator, a rodenticide, a desiccant, a nutrient and any suitable carrier disclosed in U.S. Pat. Nos. 6,303,039 B1; 7,485,451; 10,932,469 B2; EP 0818135, CA 1229497, WO 2013090628, EP 0192342, WO 2008103422, and CA 1041788.

### Some Selected Definitions:

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in microbiology, molecular biology and medicine can be found, for example, in The Merck Manual of Diagnosis and Therapy, 19th Edition, published by Merck Sharp & Dohme Corp., 2011 (ISBN 978-0-911910-19-3); Robert S. Porter et al. (eds.), Black, Jacquelyn G. Microbiology: Principles and Explorations, 9th Edition: Wiley; 9^{th} Edition, 2014, Moore, Veranus A. Principles of Microbiology: A Treatise on Bacteria, Fungi and Protozoa: Forgotten Books, 2012, The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level.

The terms “increased” ,“increase” or “enhance” or “activate” are all used herein to generally mean an increase of a property, level, or other parameter by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

As used herein, the term “pharmaceutically acceptable carrier” can include any material or substance that, when combined with an active ingredient (e.g., microbial consortium), allows the ingredient to retain biological activity and is non-reactive with the subject's immune system. Examples include, but are not limited to, any of the standard pharmaceutical carriers such as a phosphate buffered saline solution, emulsions such as oil/water emulsion, and various types of wetting agents.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Other terms are defined within the description of the various aspects and embodiments of the technology of the following.

All references cited herein are incorporated by reference in their entirety as though fully set forth.

One skilled in the art will recognize many methods and materials similar or equivalent to those described herein, which could be used in the practice of the present invention. Indeed, the present invention is in no way limited to the methods and materials described.

The technology described herein is further illustrated by the following examples which in no way should be construed as being further limiting.

Some Embodiments of the Assays, Methods, and Compositions described herein can be Defined According to any of the Following Numbered Paragraphs:
1. An assay, comprising:
   (a) culturing a population of microorganisms in a minimal media comprising nitrogen and one or more carbon molecules;
   (b) capturing a series of images of the microorganisms; and
   (c) processing the series of images, wherein processing the series of images identifies one or more phenotypes of interest in the population of microorganisms.
2. The assay of paragraph 1, wherein prior to step (a), the population of microorganisms were subjected to mutagenesis prior to culturing.
3. The assay of paragraph 2, wherein the mutagenesis comprises random mutagenesis.
4. The assay of paragraph 2, wherein the mutagenesis comprises homologous recombination.
5. The assay of any one of paragraphs 1-4, wherein the one or more carbon molecules comprises a plastic.
6. The assay of any one of paragraphs 1-5, wherein the one or more carbon molecules comprises a perfluoroalkyl (PFA) chemical.
7. The assay of any one of paragraphs 1-6, wherein capturing a series of images of the microorganisms is over a period of at least 1 week.
8. The assay of any one of paragraphs 1-7, wherein processing step (c) comprises applying a calibration curve generated by a second-degree polynomial curve from two or more of: a raw image measurement, an optical density; and a cell density value.
9. The assay of any one of paragraphs 1-8, wherein the one or more phenotypes of interest in the population of microorganisms comprises a growth curve.
10. The assay of any one of paragraphs 1-9, wherein the one or more phenotypes of interests comprises plastic degradation or PFA degradation.
11. The assay of paragraph 5, wherein the PFA chemical is selected from the group consisting of: Polyethylene (PE), Polyethylene Terephthalate (PET), Polyvinyl Chloride (PVC), Polypropylene (PP), Polystyrene (PS), Polylactide Acid (PLA), Polycarbonate (PC), Acrylic (PMMA), Acetal (Polyoxymethylene, POM), Nylon (PA), ABS (Acrylonitrile Butadiene Styrene).
12. The assay of paragraph 6, wherein the PFA chemical is selected from the group consisting of: Perfluorooctanoic acid (PFOA), Perfluoroctanesulfonic acid (PFOS), Perfluorohexane sulfonate (PFHxS), and Perfluorononanoic acid (PFNA).
13. A composition for waste degradation, the composition comprising:
   (a) a preparation of a microbial consortium of microorganisms that possess one or more phenotypes of interest identified by the assay of claim 1; and
   (b) a carrier.
14. The composition of paragraph 13, wherein the one or more phenotypes of interest comprise plastic degradation or PFA chemical degradation.
15. The composition of any one of paragraphs 13-14, wherein the microorganisms are a population of bacterium.
16. The composition of any one of paragraphs 13-15, wherein the carrier is selected from the group consisting of: a culture medium, a pharmaceutically acceptable carrier, a gel, a powder, a soil, a compost pellet, a fertilizer, a tablet, a solid support.
17. A method for selecting a microorganism for the degradation of waste, the method comprising:
   (a) culturing a pre-selected population of microorganisms in a cell culture well, wherein the cell culture well comprises a minimal media comprising a PFA chemical or a plastic;
   (b) capturing a series of images of the cell culture well;
   (c) processing the series of images from step (b), wherein processing the series of images determines an optical density; and
   (d) when the optical density of the cell culture increases over a period of time, selecting the population of microorganisms for degradation of waste.
18. The method of paragraph 17, prior to step (a), subjecting the pre-selected population of microorganisms to mutagenesis.
19. The method of paragraph 17, wherein the pre-selected population of microorganisms are the same species or strain of microorganisms.
20. The method of paragraph 17, wherein the pre-selected population of microorganisms are different species or strains of microorganisms.
21. A culture of a population of microorganisms possessing the one or more phenotypes of interest identified by the assay of any one of paragraphs 1-12.
22. A biological agent isolated from the population of microorganisms of any one of paragraphs 13-16 or 21.
23. The biological agent of paragraph 22, wherein the biological agent is a peptide or protein.
24. The biological agent of paragraph 23, wherein the biological agent is a nucleic acid.
25. A medium, comprising:
   nitrogen and one or more carbon molecules.
26. The medium of paragraph 25, wherein the one or more carbon molecules is selected from the group consisting of: Perfluorooctanoic acid (PFOA), Perfluoroctanesulfonic acid (PFOS), Perfluorohexane sulfonate (PFHxS), and Perfluorononanoic acid (PFNA).
27. A system comprising:
   (a) a reservoir for containing a population of microorganisms in a minimal media, wherein the minimal media comprises nitrogen and one or more carbon molecules;
   (b) a camera capable of capturing a series of images of the microorganisms; and
   (c) a data processing apparatus.

### EXAMPLES

### Example 1

### Directed Evolution—Assay Format

The Inventors' assay works by growing our microorganism on minimal media supplemented with the carbon source of interest. Minimal media only contains trace salts and a nitrogen source, but no carbon source.

As microorganisms require a carbon source to grow, minimal media is supplement the target carbon source one seeks to metabolize. This carbon source can be plastic, PFAS chemicals, or other difficult to break down chemicals.

The carbon source in powder form with average particle size of 40-50 micrometers if the carbons source is hydrophobic or dissolve it if it is soluble in water. As, not all of the organisms can readily utilize these carbon sources, this exerts evolutionary selective pressure, that one can then use to select for organisms with the desired metabolic properties.

One can introduce random or recombination based mutagenesis in order to increase genetic diversity. This includes radiation, or various error prone or homologous nucleic acids introduced into phage libraries for introduction to microorganisms.

### Example 2

### Image Processing Pipeline

Images taken on certain automation systems (e.g., Opentron robots) are slightly off angle due to the attached camera being installed at an angle. The first part of our software converts the single image of the deck into flat facing images of each of the 11 bays on the robot. The perspective adjusted images have a significant amount of empty space outside of the petri dish. The second part of the software crops out only the portion of the petri dish for further analysis.

The cropped images are measured using the ImageJ/FIJI. This raw image measurement value is very sensitive to changes in the petri dish that result from growth of microorganisms or changes in lighting. Image measurement are converted to optical density or cell density measurement through a calibration curve. The curve is generated by measuring known optical density values in a petri dish in each bay of the robot to yield a corresponding raw image measurement value. Calibration curve is then constructed by plotting the raw image measurement values (x values) against the optical density or cell density values (y values) and then fitting these points with a second-degree polynomial curve. The resulting function therefore takes in raw image measurement values and converts them to optical. The cropped images are measured using the ImageJ/FIJI. This raw image measurement value is very sensitive to changes in the petri dish that result from growth of microorganisms or changes in lighting. Image measurement are converted to optical density or cell density measurement through a calibration curve. The curve is generated by measuring known optical density values in a petri dish in each bay of the robot to yield a corresponding raw image measurement value. Calibration curve is then constructed by plotting the raw image measurement values (x values) against the optical density or cell density values (y values) and then fitting these points with a second-degree polynomial curve. The resulting function therefore takes in raw image measurement values and converts them to optical density or cell density values. The calibration curve/function is specific for each bay due to the effect of lighting on the raw image measurement.

Parallel processing is used to speed the processing of the thousands of images created during the experiment. Final Data is stored in a SQL database and the time series data generated from the image processing pipeline is matched to experimental conditions and organism number to allow for an organized and efficient manner to store and access all experimental data.

Described herein is an assay, including culturing a population of microorganism in a minimal media including nitrogen and one or more carbon molecules; capturing a series of images of the microorganisms; and processing the series of images, wherein processing the series of images identifies one or more phenotypes of interest in the population of microorganisms. In other embodiments, the population of microorganisms was subjected to mutagenesis prior to culturing. In other embodiments, mutagenesis comprises random mutagenesis. In other embodiments, mutagenesis homologous recombination. This includes, for example, mutagenesis methods, such as site-directed mutagenesis including CRISPR cas systems, zinc finger nucleases (ZFNs), transcription activator-like effector nucleases (TALENs), meganucleases. Examples of random:mutatgenesis includes error-prone PCR, rolling circle error-prone PCR, mutator strains deficient in DNA repair mechanisms, temporary mutator strains with overexpressed mutator alleles, insertion mutagenesis where codons are randomly inserted in genes of interested to generate diversity, ethyl methanesulfonate, Nitrous acid, DNA shuffling, or UV radiation. As ready understood by one of ordinary skill, the method can include multiple rounds of mutagenesis, wherein after identifying a phenotype of interest, that mutated population possessing the phenotype of interest, is then subject to one, two, three or more additional rounds of mutagenesis to create genetic diversity, with additional rounds of selection, and analysis, thereby directing evolution by continued and repeated application of selection pressure. Here, that includes minimal media with carbon source intake and capability to metabolize these products. In other embodiments, the one or more carbon molecules comprises plastic. In other embodiments, the one or more carbon molecule comprises PFAS. In other embodiments, capturing a series of images of the microorganisms is over a period of at least 1 week. In other embodiments, processing the series of images comprises applying a calibration curve generated by a second-degree polynomial curve from two or more of: raw image measurement, optical density and cell density values. In other embodiments, the one or more phenotypes of interest in the population of microorganisms comprises a growth curve. In other embodiments, the one or more phenotypes of interests comprises plastic or PFAS degradation.

Also described herein is a culture of a population of microorganisms possessing the one or more phenotypes of interest identified by the assay culturing a population of microorganism in a minimal media including nitrogen and one or more carbon molecules; capturing a series of images of the microorganisms; and processing the series of images, wherein processing the series of images identifies one or more phenotypes of interest in the population of microorganisms. Bacteria microorganism examples include Acidimicribioum sp. Strain A6, Acidovorax sp., Alcaligens Faecalis. Amycolatopsis sp., Bacillus Subtilis, Bacillus, Brevibacillus borstelensis, Caenibacterium thermophilum, Clostridium acetobutylicum, Clostridium botulinum, Corynebacterium Comamonas acidovorans, Clostridium botulinum, Dehalococcoides Diplococcus sp., Enterobacter, Enterobacter agglomerans, Escherichia, E. coli, Enterococci Ideonella sakaiensis, Lactobacillus, Micrococcus sp., Moraxella sp., Nocardia farcinica, Nitrobacter spp., Nitrosomonas spp., Ochrobactrum, Pichia Pastoris, Pseudomonas, Pseudomonas chlororaphis, Pseudomonas putida, Pseudomonas indica, Pseudomonas lemoignei, Pseudomonas stutzeri, Rhodococcus, Ralstonia pikettii, Salmonella, Staphylococcus, Streptococcus, Streptomyces, Shigella, Schlegelella thermodepolymerans, Thiobacillus spp., Listeria, Firmicutes, and Proteobacteria.

Fungi examples include Aspergillus, Penicillium, Pullularia, Podospora, Neurospora, Trichoderma, and Chaetomium

The platform is widely compatible, so long as the organism possess a common doubling rate on the order of minutes and when cultured in minimal media with alternative carbon source is viable with a population doubling time on the order of hours or days.

Also described herein is a biological agent isolated from the population of microorganisms possessing the one or more phenotypes of interest identified by the assay culturing a population of microorganism in a minimal media including nitrogen and one or more carbon molecules; capturing a series of images of the microorganisms; and processing the series of images, wherein processing the series of images identifies one or more phenotypes of interest in the population of microorganisms.

In other embodiments, the biological agent is a peptide or protein. In other embodiments, the biological agent is a nucleic acid.

Described herein is a media. In various embodiments, the media includes one or more of: one or more trace salts, one or more nitrogen sources and one or more carbon molecules.

In various embodiments, the media includes one or more trace salts. In various embodiments, trace salts can include Disodium Phosphate (sodium phosphate dibasic), Monopotassium phosphate (potassium phosphate monobasic), Sodium Chloride, Calcium Chloride. In various embodiments, trace salts can also include Copper Sulfate Pentahydrate, Ferric Citrate, Ferric Chloride (Iron(III) Chloride), Ferric Sulfate (Iron (III) Sulfate), Magnesium Sulfate Heptahydrate, Manganese Sulfate Monohydrate, Potassium Chloride, Dipotassium phosphate, Vitamin B1 (thiamine), Sodium Acetate, Sodium Molybdate Dihydrate, Sodium Nitrate, Sodium Tetraborate, Zinc sulfate Heptahydrate, p-Aminobenzoic acid, Niacin, Pyridoxin HCl, Riboflavin, Thiamin HCl, Choline HCL, d-Biotin (or Biotin in general), Adenine HCL L-Methionine, L-Lysine or Arginine, Riboflavin, Yeast Extract, Peptone, Tryptone, Malt Extract.

In various embodiments, the media includes one or more nitrogen sources. In various embodiments, nitrogen sources includes Ammonium Chloride, Ammonium Sulphate, Ammonium Nitrate, Yeast Extract, Peptone, Nutrient Broth, Meat Extract and Tryptone

In various embodiments, the media includes one or more plastics. Examples include, Polyethylene (PE), Polyethylene Terephthalate (PET), Polyvinyl Chloride (PVC), Polypropylene (PP), Polystyrene (PS), Polylactide Acid (PLA), Polycarbonate (PC), Acrylic (PMMA), Acetal (Polyoxymethylene, POM), Nylon (PA), ABS (Acrylonitrile Butadiene Styrene). Generally, any crystalline polymer that is durable (e.g., high melting point, etc) derived from crude oil is compatible with the platform. In various embodiments, the one or more carbon sources is in a film, nurdles or powder. In various embodiments, the one or more carbon sources include a perfluoroalkyl (PFA) chemical, which includes Perfluorooctanoic acid (PFAS), Perfluoroctanesulfonic acid (PFOS), Perfluorohexane sulfonate (PFHxS), Perfluorononanoic acid (PFNA)

In various embodiment, the media is included in a fed-batch system where the feed contains the media components or carbon sources mentioned. In various embodiments, the media includes a precursor molecule of plastic or PFAS with our without the PFAS chemical also in solution.

### Example 3

### Microorganisms

Future starter organisms may include by are not limited to Pseudomonas putida, Pichia pastoris. Generally speaking, any number of microorganisms would be compatible with this process, including those listed herein. An important criterion is simply that normal growth rate is on the order of minutes and when placed on the minimal media with alternative carbon source, proliferation then slows and occurs instead on the order of hours or days.

### Example 4

### Minimal Media

Minimal media contains one or more trace salts such as Disodium Phosphate (sodium phosphate dibasic), Monopotassium phosphate (potassium phosphate monobasic), Sodium Chloride, Calcium Chloride, one or more nitrogen sources such as Ammonium Chloride, Ammonium Sulphate, Ammonium Nitrate, Yeast Extract, Peptone, Nutrient Broth, Meat Extract and Tryptone, and the target carbon source of interest including perfluoroalkyl (PFAS) chemicals such as a Perfluorooctanoic acid (PFAS), Perfluoroctanesulfonic acid (PFOS), Perfluorohexane sulfonate (PFHxS), and/or Perfluorononanoic acid (PFNA).

For a target carbon source of interest, generally, any crystalline polymer that is durable (e.g., high melting point, etc) derived from crude oil is compatible with the platform. In various embodiments, the one or more carbon sources is in a film, nurdles or powder. In some instances, where the carbon source is in powder form, there is an average particle size of 40-50 micrometers if the carbons source is hydrophobic or dissolve it if it is soluble in water

### Example 5

### Applications—Assay Screening

The use of organisms with any level of activity for plastic degradation and or assimilation as a starter organism for directed evolution. In an example, one can culture microorganism in a minimal media including nitrogen and one or more carbon molecules, capturing a series of images of the microorganisms, and processing the series of images.

### Example 6

### Applications—Assay Screening with Mutagenesis

The use of organisms with any level of activity for plastic degradation and or assimilation as a starter organism for directed evolution. In an example, one can culture microorganism in a minimal media including nitrogen and one or more carbon molecules, capturing a series of images of the microorganisms, and processing the series of images, with mutagenesis prior to culturing. Mutagenesis can include techniques with random mutagenesis (e.g., radiation, chemical additives, error prone polymerases, or homologous recombination.

One can apply multiple rounds of mutagenesis, wherein after identifying a phenotype of interest, that mutated population possessing the phenotype of interest, is then subject to one, two, three or more additional rounds of mutagenesis to create genetic diversity, with additional rounds of selection, and analysis, thereby directing evolution by continued and repeated application of selection pressure.

### Example 7

### Organisms and Media Compositions

The table below (Table 3) summarizes the starter microorganisms tested on the fully automated system using the assay described above and the carbon sources used in the minimal media. A positive result indicated that the microorganism was capable of carbon source degradation. Microorganisms subjected to UV mutagenesis are indicated below.

TABLE 3
Summary of Carbon Source Degradation Results

| | Carbon Source(s) | Carbon source |
|---|---|---|
| Starter Microorganism Name | in the | degradation? |
| (Source, Modifications) | minimal media | +/− |
| E. coli (Genspace) | Ethylene Glycol | + |
| E. coli (Genspace) | PET pellet | + |
| E. coli (Genspace, | Polyethylene powder | − |
| UV Mutagenesis) | | |
| E. coli (Genspace, | Glucose | + |
| UV Mutagenesis) | | |
| P. fluorescens (Genspace) | Glucose | − |
| P. fluorescens (Genspace) | Ethylene Glycol | + |
| P. fluorescens (Genspace) | Polyethylene powder | + |
| P. fluorescens (Genspace) | PFNA Solution | + |
| P. fluorescens (ATCC) | PFNA Solution | + |
| P. fluorescens (ATCC) | PFAS absorbed on | − |
| | GAC | |
| P. fluorescens, P. aeruginosa | PFAS absorbed on | − |
| (ATCC) | GAC | |
| P. fluorescens, P. aeruginosa | PFNA Solution | + |
| (ATCC) | | |

Solution compositions are provided below for the general M9 and PFNA stock solutions used in the Examples above.

M9 Stock Solution

| Component | Addition | |
|---|---|---|
| Sodium Phosphate Dibasic | 15 | g |
| Potassium Phosphate Monobasic | 7.5 | g |
| Ammonium Chloride | 2.5 | g |
| Sodium Chloride | 1.25 | g |
| Calcium Chloride | 9 | mg |
| DI water | 1 | Liter |

DI water is added to a final volume of 1 L. Autoclaved for 15 min @ 121 C.

PFNA Stock Solution

| Component | Addition | |
|---|---|---|
| Sodium Phosphate Dibasic | 0.120 | g |
| DI water | 1 | Liter |

### Example 8

### Summary of LC-MS Chemical Analysis of Microbial PFAS Degradation

### Time-Lapse Image Analysis/Carbon Source Assays:

The method provided herein can be used to identify novel bacteria with the ability to degrade environmental contaminants made of carbons such as plastics, PFAS chemicals, chlorinated solvents, and the like. This method can identify useful microorganisms for waste degradation cheaply but also in a high-throughput manner which is a major advantage over traditional carbon source assays. The carbon source assay provided here functions by growing bacteria in M9 media described above where the only carbon source available is the contaminant of interest. Changes in the turbidity of the solution is an indication of growth and by extension an indication of catabolism for the carbon source of interest.

It is important to note that growth rates on alternative carbon sources are much slower than typical carbon sources like glucose. In order to run this assay for plastics and PFAS, careful monitoring for weeks or months is required. In order to make this entire process efficient, automated imaging of the experiments over long periods of time can be conducted and images are processed using proprietary software to reveal the desired change in turbidity.

### Liquid Chromatography-Mass Spectrometry (LC-MS) Studies:

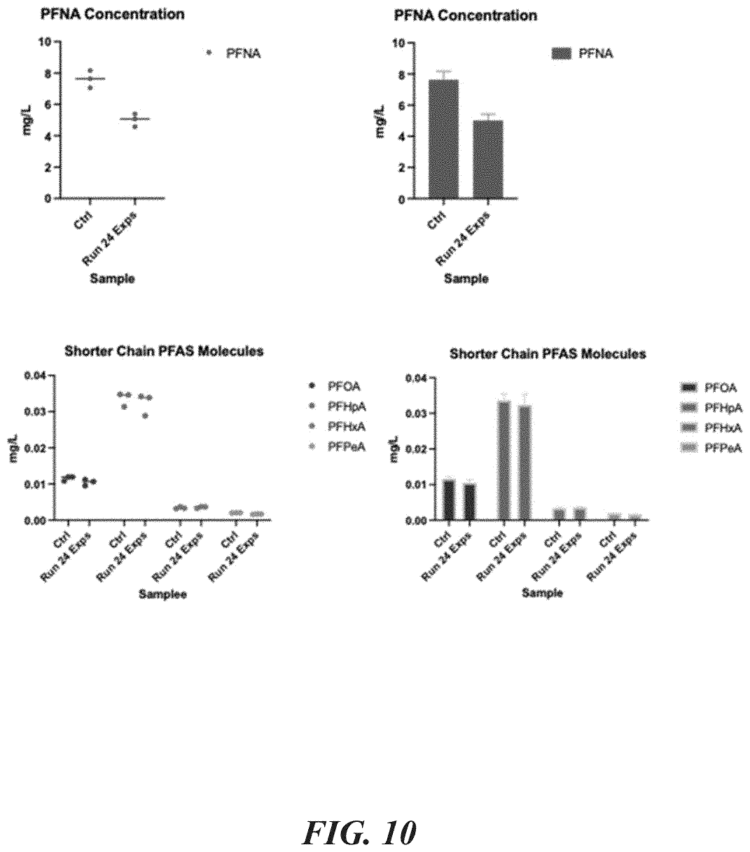

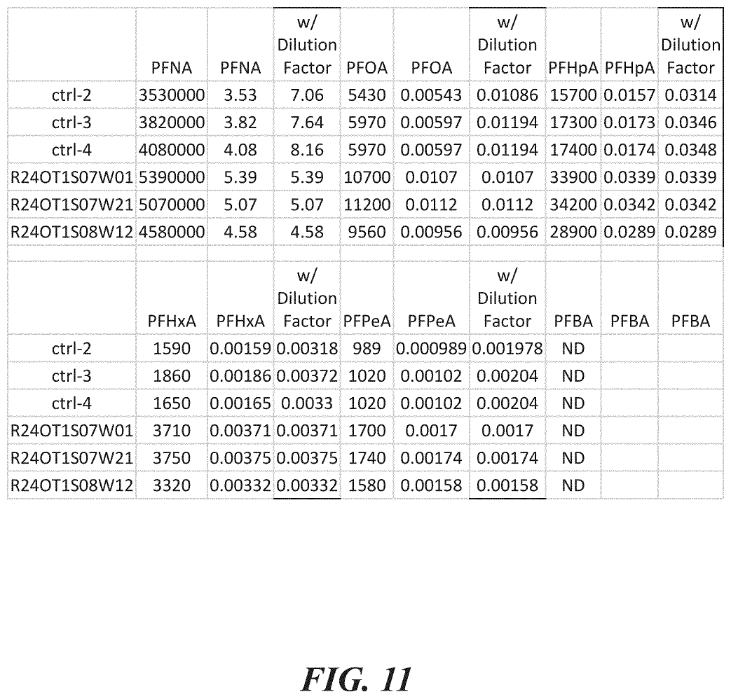

The growth of bacteria on alternative carbon sources is an indirect indicator that the carbon source is being degraded/utilized by the bacteria. In order to confirm degradation and also assess the degradation rate of the carbon source in comparison with growth rate, it was necessary to validate the Inventor's system, assay, and methodologies using direct measurements of the carbon source over time. For water-soluble molecules like PFAS, LC-MS is an ideal solution to determine PFAS concentration over time. The technical details of the experiment are listed below and the results are provided in FIGS. 10 and 11.

Run 24 Experiment Setup
5× Dilution of M9 Stock
10× Dilution of PFNA stock solution
To the inventors added:
2 mL diluted M9 media
50 μL diluted PFNA solution
200 μL cells (P. fluorescens, P. aeruginosa) from overnight
To Controls the inventors add:
1.2 ml M9
25 μL PFNA
One month+ of incubation with regular data collection

Data Collection
Images of both plates every two hours
Run 24 Vista LC-MS Time point 1
Run 24 Vista LC-MS Time point 2
Run 24 Vista LC-MS Time point 3
Run 24 Vista LC-MS Time point 4
Assess average degradation rate (>10 samples)
Run 24 Image Processing Results
Run 24 sequencing Results

Vista results so far have revealed significant changes in PFNA concentration after incubation with Pseudomonas strains (FIGS. 10-11). Therefore, the automated assay and methods provided herein are successful for predicting microbial degradation of PFA chemicals.

### Example 9

### Time Course of the Assay

Below is a general time course of the assay provided herein:
Experimental Setup—Automated, approximately 10 mins
Run Time—Approximately 1-2 months depending on the carbon source with continuous automated monitoring/imaging
Time-lapse Sampling—Automated, every 2 hours

### Example 10

### Applications-Fermentation

Once a novel microorganism is identified by the assay described above, established fermentation techniques can be used. This scale-up process can utilize a bioreactor as a vessel to carry out the biochemical reaction with carbon source at a concentration suitable for the size of the vessel and/or bioprocess being utilized. The carbon source and other nutrients in the media can be supplied in a batch, fed-batch, or continuous manner. Furthermore, the vessel environmental conditions (flow rates, temperatures, pH, dissolved oxygen, agitation speed, etc.) can be monitored and controlled automatically.

### Conclusion

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the compositions and methods related to microorganism generation, including by directed evolution or otherwise, plastics and similar materials ingested by such microorganisms, methods and compositions related to use of the aforementioned compositions, techniques and composition and use of solutions used therein, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Marküsh groups used in the appended claims.

Preferred embodiments of this invention are described herein, including the best mode known to the inventor for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

What is claimed is:

1. An assay, comprising:
(a) culturing a population of microorganisms in a minimal media comprising nitrogen and one or more carbon molecules;
(b) capturing a series of images of the microorganisms; and
(c) processing the series of images, wherein processing the series of images identifies one or more phenotypes of interest in the population of microorganisms.

2. The assay of claim 1, wherein prior to step (a), the population of microorganisms were subjected to mutagenesis prior to culturing.

3. The assay of claim 2, wherein the mutagenesis comprises random mutagenesis.

4. The assay of claim 2, wherein the mutagenesis comprises homologous recombination.

5. The assay of claim 1, wherein the one or more carbon molecules comprises a plastic.

6. The assay of claim 1, wherein the one or more carbon molecules comprises a perfluoroalkyl (PFA) chemical.

7. The assay of claim 1, wherein capturing a series of images of the microorganisms is over a period of at least 1 week.

8. The assay of claim 1, wherein processing step (c) comprises applying a calibration curve generated by a second-degree polynomial curve from two or more of: a raw image measurement, an optical density; and a cell density value.

9. The assay of claim 1, wherein the one or more phenotypes of interest in the population of microorganisms comprises a growth curve.

10. The assay of claim 1, wherein the one or more phenotypes of interests comprises plastic degradation or PFA degradation.

11. The assay of claim 5, wherein the PFA chemical is selected from the group consisting of:
Polyethylene (PE), Polyethylene Terephthalate (PET), Polyvinyl Chloride (PVC), Polypropylene (PP), Polystyrene (PS), Polylactide Acid (PLA), Polycarbonate (PC), Acrylic (PMMA), Acetal (Polyoxymethylene, POM), Nylon (PA), ABS (Acrylonitrile Butadiene Styrene).

12. The assay of claim 6, wherein the PFA chemical is selected from the group consisting of: Perfluorooctanoic acid (PFOA), Perfluoroctanesulfonic acid (PFOS), Perfluorohexane sulfonate (PFHxS), and Perfluorononanoic acid (PFNA).

13. A composition for waste degradation, the composition comprising:
(a) a preparation of a microbial consortium of microorganisms that possess one or more phenotypes of interest identified by the assay of claim 1; and
(b) a carrier.

14. The composition of claim 13, wherein the one or more phenotypes of interest comprise plastic degradation or PFA chemical degradation.

15. The composition of claim 13, wherein the isolated microorganisms are a population of bacterium.

16. The composition of claim 13, wherein the carrier is selected from the group consisting of: a culture medium, a pharmaceutically acceptable carrier, a gel, a powder, a soil, a compost pellet, a fertilizer, a tablet, a solid support.

17. A method for selecting a microorganism for the degradation of waste, the method comprising:
(a) culturing a pre-selected population of microorganisms in a cell culture well, wherein the cell culture well comprises a minimal media comprising a PFA chemical or a plastic;
(b) capturing a series of images of the cell culture well;
(c) processing the series of images from step (b), wherein processing the series of images determines an optical density; and
(d) when the optical density of the cell culture increases over a period of time, selecting the population of microorganisms for degradation of waste.

18. The method of claim 17, prior to step (a), subjecting the pre-selected population of microorganisms to mutagenesis.

19. The method of claim 17, wherein the pre-selected population of microorganisms are the same species or strain of microorganisms.

20. The method of claim 17, wherein the pre-selected population of microorganisms are different species or strains of microorganisms.
